(19) United States
(12) Patent Application Publication
(10) Pub. No.: US 2025/0272460 A1
(43) Pub. Date: Aug. 28, 2025

(54) SIMULATION OF MINERALIZATION IN ROCK POROUS MEDIA BY STATISTICAL SAMPLING OF NUCLEATION SITES

(71) Applicant: International Business Machines Corporation, Armonk, NY (US)

(72) Inventors: JAIONE TIRAPU AZPIROZ, Rio de Janeiro (BR); David Alejandro Lazo Vasquez, São Paulo (BR); Matheus Esteves, Rio de Janeiro (BR); Rodrigo Neumann Barros Ferreira, Rio de Janeiro (BR); Ademir Ferreira da Silva, São Paulo (BR); Mathias B. Steiner, Rio de Janeiro (BR)

(21) Appl. No.: 18/590,891

(22) Filed: Feb. 28, 2024

(51) Int. Cl. G06F 30/28

(52) U.S. Cl. CPC G06F 30/28

(57) ABSTRACT

A representation of a rock capillary network is obtained and initial and boundary conditions of fluid flow and mineral precipitation process simulations for the rock capillary network are set. One or more instances of a geometry evolution simulation are performed, each geometry evolution simulation comprising obtaining fluid flow vectors for the rock capillary network, identifying one or more hotspots of nucleation in the rock capillary network, starting a mineral precipitation analysis and estimating a mineral accumulation over a given time interval for at least the identified hotspots of nucleation, adjusting a pore geometry to an effect of mineral precipitation for the rock capillary network, and iteratively repeating each of the one or more geometry evolution simulations until corresponding stop criteria are met for each geometry evolution simulation. A resulting rock under analysis property is computed from an aggregate of results of the one or more geometry evolution simulations.

### BACKGROUND

The present invention relates generally to the electrical, electronic and computer arts and, more particularly, to computer-aided materials science, computational fluid mechanics, and the like.

Computer simulators are available for computing the impact of fluid-rock processes on the pore scale geometry. Applications include, for example, reservoir engineering, oil recovery, the growing field of carbon dioxide geological sequestration, and the like. Typically, such computer simulators serve to quantify the influence of injection and flow of carbon dioxide (CO₂) inside a capillary network representative of the rock under analysis, as well as the geometry modification of rock capillaries, often applying deterministic models for physical property changes and chemical reactions to estimate geological CO₂ mineralization and storage.

### BRIEF SUMMARY

Principles of the invention provide techniques for simulation of mineralization in rock porous media by statistical sampling of nucleation sites. In one aspect, an exemplary method includes the operations of obtaining a representation of a rock capillary network; setting initial and boundary conditions of fluid flow and mineral precipitation process simulations for the rock capillary network; performing one or more instances of a geometry evolution simulation, each geometry evolution simulation comprising obtaining fluid flow vectors for the rock capillary network, identifying one or more hotspots of nucleation in the rock capillary network, starting a mineral precipitation analysis and estimating a mineral accumulation over a given time interval for at least the identified hotspots of nucleation, adjusting a pore geometry to an effect of mineral precipitation for the rock capillary network, and iteratively repeating each of the one or more geometry evolution simulations until corresponding stop criteria are met for each geometry evolution simulation; and computing a resulting rock under analysis property from an aggregate of results of the one or more geometry evolution simulations.

In one aspect, a computer program product comprises one or more tangible computer-readable storage media and program instructions stored on at least one of the one or more tangible computer-readable storage media, the program instructions executable by a processor, the program instructions comprising obtaining a representation of a rock capillary network; setting initial and boundary conditions of fluid flow and mineral precipitation process simulations for the rock capillary network; performing one or more instances of a geometry evolution simulation, each geometry evolution simulation comprising obtaining fluid flow vectors for the rock capillary network, identifying one or more hotspots of nucleation in the rock capillary network, starting a mineral precipitation analysis and estimating a mineral accumulation over a given time interval for at least the identified hotspots of nucleation, adjusting a pore geometry to an effect of mineral precipitation for the rock capillary network, and iteratively repeating each of the one or more geometry evolution simulations until corresponding stop criteria are met for each geometry evolution simulation; and computing a resulting rock under analysis property from an aggregate of results of the one or more geometry evolution simulations.

In one aspect, an apparatus comprises a memory and at least one processor, coupled to the memory, and operative to perform operations comprising obtaining a representation of a rock capillary network; setting initial and boundary conditions of fluid flow and mineral precipitation process simulations for the rock capillary network; performing one or more instances of a geometry evolution simulation, each geometry evolution simulation comprising obtaining fluid flow vectors for the rock capillary network, identifying one or more hotspots of nucleation in the rock capillary network, starting a mineral precipitation analysis and estimating a mineral accumulation over a given time interval for at least the identified hotspots of nucleation, adjusting a pore geometry to an effect of mineral precipitation for the rock capillary network, and iteratively repeating each of the one or more geometry evolution simulations until corresponding stop criteria are met for each geometry evolution simulation; and computing a resulting rock under analysis property from an aggregate of results of the one or more geometry evolution simulations.

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor, oil field equipment, carbon dioxide sequestration equipment, or the like, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects, as will be discussed further below. Features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1A is a high-level block diagram of an example system for simulation of mineralization in rock porous media by statistical sampling of nucleation sites, in accordance with an example embodiment;

FIG. 1B is a high-level block diagram of an example geometry evolution simulator sub-system for computing the impact of fluid-rock processes on the pore scale geometry, in accordance with an example embodiment.

FIG. 1C is an example of a rock capillary network representation at two instants in a simulation, in accordance with an example embodiment;

FIG. 1D is a representation of an example distribution of pore diameters within a rock capillary network representation at two instants in the simulation, in accordance with an example embodiment;

FIG. 2 is a high-level block diagram of a plurality of geometry evolution simulator sub-systems and a rock capillary network property estimator, in accordance with an example embodiment;

FIG. 3 is a flowchart of an example method for simulation of mineralization in rock porous media by statistical sampling of nucleation sites, in accordance with an example embodiment;

FIG. 4 is a flowchart showing details of operations of the method of FIG. 3, in accordance with an example embodiment;

FIG. 5 is a flowchart showing details of operations of the method of FIG. 3, in accordance with an example embodiment;

FIG. 6 is a flowchart showing details of operations of the method of FIG. 3, in accordance with an example embodiment;

FIG. 7A is a representation of a rock capillary network, in accordance with an example embodiment;

FIG. 7B is a table providing details regarding the nodes of FIG. 7A and the links (capillaries) between the nodes of FIG. 7A, in accordance with an example embodiment;

FIG. 8 is a detailed representation of the rock capillary network illustrating the flow from node 1 to node 2, in accordance with an example embodiment;

FIG. 9A is a detailed representation of the rock capillary network illustrating the location of nodes 3-7 and the flow from node 1 to node 2, in accordance with an example embodiment;

FIG. 9B is a table showing details of the nodes and links (capillaries) within the rock capillary network, in accordance with an example embodiment;

FIG. 10A illustrates example randomly selected nucleation hotspots in the rock capillary network, in accordance with an example embodiment;

FIG. 10B is a table providing details for the randomly selected nucleation hotspots of FIG. 10A, in accordance with an example embodiment;

FIGS. 11A and 11B are tables providing details regarding fluid flow, geometry, and pore-scale process parameters that may trigger the onset of nucleation sites within the rock capillary network, in accordance with an example embodiment;

FIGS. 12A-12B illustrate a representation of the rock capillary network and parameters used to simulate the physical properties and chemical reactions of fluid flow on capillaries, in accordance with an example embodiment;

FIG. 12C is a table illustrating fluid flow and rock capillary geometrical characterization, in accordance with an example embodiment;

FIG. 13 illustrates two parallel simulation instances, each with a randomly selected sub-set of nucleation hotspots where mineral precipitation is assumed to occur and where the capillary geometry of the rock capillary network is changed, in accordance with an example embodiment;

FIG. 14A is a three-dimensional rendering illustrating the typical rock capillary diameter values encountered during the flow simulation, used as an example to show the geometrical characteristics of the capillaries in determining nucleation hotspots leading to consequent onset mineralization, in accordance with an example embodiment;

FIG. 14B is a three-dimensional rendering illustrating the typical pressure values on rock capillaries encountered during the flow simulation, showing the influence of pressure fields on the rock capillaries and in determining hotspots of nucleation leading to the consequent onset mineralization, in accordance with an example embodiment;

FIG. 14C is a table illustrating an example of flow simulation parameters on a rock capillary network, including fluid flow and capillary geometry, in accordance with an example embodiment;

FIG. 14D is a three-dimensional representation of the capillary local inclination, in accordance with an example embodiment;

FIG. 15 is an overview of an example system architecture for performing the simulation of mineralization in rock porous media, in accordance with an example embodiment; and

FIG. 16 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

Given the discussion herein (reference characters refer to the drawings discussed below), it will be appreciated that, in general terms, an exemplary method for performing a stochastic simulation of mineral precipitation and mineral accumulation on a rock pore-scale, according to an aspect of the invention, includes the operations of obtaining a representation of a rock capillary network 232 (operation 304); setting initial and boundary conditions of fluid flow and mineral precipitation process simulations for the rock capillary network 232 (operation 308); performing one or more instances of a geometry evolution simulation, each geometry evolution simulation comprising obtaining fluid flow vectors for the rock capillary network 232 (operation 312), identifying one or more hotspots of nucleation in the rock capillary network 232 (operation 316), starting a mineral precipitation analysis and estimating a mineral accumulation over a given time interval 276 for at least the identified hotspots of nucleation (operation 320), adjusting a pore geometry to an effect of mineral precipitation for the rock capillary network 232 (operation 324), and iteratively repeating each of the one or more geometry evolution simulations until corresponding stop criteria are met for each geometry evolution simulation (operation 326); and computing a resulting rock under analysis property from an aggregate of results of the one or more geometry evolution simulations (operation 328). These features provide the technical benefits of computing the impact of fluid-rock processes on the pore scale geometry; enabling the prediction, within a capillary network representation framework of a rock sample, the spatiotemporal geometry evolution of the porous structure resulting from pore-scale processes; a system for estimating the volume of carbon dioxide converted and stored at a large scale, such as on the scale of a reservoir, based on pore-scale processes; supporting the generation of new additive candidates through simulation to accelerate mineralization under geological conditions; and a process for the computation of the porous geometry evolution that models the probabilistic nature of the onset of nucleation to identify a set of most likely nucleation spots (referred to as nucleation hotspots or simply hotspots herein), referring to those capillaries in the network where precipitation will happen.

In one aspect, a computer program product comprises one or more tangible computer-readable storage media and program instructions stored on at least one of the one or more tangible computer-readable storage media, the program instructions executable by a processor, the program instructions comprising obtaining a representation of a rock capillary network 232 (operation 304); setting initial and boundary conditions of fluid flow and mineral precipitation process simulations for the rock capillary network 232 (operation 308); performing one or more instances of a geometry evolution simulation, each geometry evolution simulation comprising obtaining fluid flow vectors for the rock capillary network 232 (operation 312), identifying one or more hotspots of nucleation in the rock capillary network 232 (operation 316), starting a mineral precipitation analysis and estimating a mineral accumulation over a given time interval 276 for at least the identified hotspots of nucleation (operation 320), adjusting a pore geometry to an effect of mineral precipitation for the rock capillary network 232 (operation 324), and iteratively repeating each of the one or more geometry evolution simulations until corresponding stop criteria are met for each geometry evolution simulation (operation 326); and computing a resulting rock under analysis property from an aggregate of results of the one or more geometry evolution simulations (operation 328).

In one aspect, a system comprises a memory and at least one processor, coupled to the memory, and operative to perform operations comprising obtaining a representation of a rock capillary network 232 (operation 304); setting initial and boundary conditions of fluid flow and mineral precipitation process simulations for the rock capillary network 232 (operation 308); performing one or more instances of a geometry evolution simulation, each geometry evolution simulation comprising obtaining fluid flow vectors for the rock capillary network 232 (operation 312), identifying one or more hotspots of nucleation in the rock capillary network 232 (operation 316), starting a mineral precipitation analysis and estimating a mineral accumulation over a given time interval 276 for at least the identified hotspots of nucleation (operation 320), adjusting a pore geometry to an effect of mineral precipitation for the rock capillary network 232 (operation 324), and iteratively repeating each of the one or more geometry evolution simulations until corresponding stop criteria are met for each geometry evolution simulation (operation 326); and computing a resulting rock under analysis property from an aggregate of results of the one or more geometry evolution simulations (operation 328).

In one example embodiment, an amount of carbon dioxide mineralized and stored in a corresponding rock capillary network representation is computed. These features provide the technical benefit of facilitating the design of carbon dioxide sequestration equipment.

In one example embodiment, estimates of converted and stored carbon dioxide are computed in a rock under analysis. These features provide the technical benefit of facilitating the design of carbon dioxide sequestration equipment.

In one example embodiment, a set of flow characteristics (e.g., backflow and reverse pressure gradients) and geometrical characteristics (e.g., throats, heels, elbows, and end capillaries) that influence a probability of nucleation are computed per capillary in the rock capillary network 232. These features provide the technical benefit of nucleation models that compute the probability of becoming a nucleation site, incorporating the effect of flow in confined spaces as in porous media, where the local geometry severely affects the fluid flow characteristics and consequent chemical reactions along the fluid paths; and nucleation models for predicting the impact of precipitation on the rock pore geometry that considers the probabilistic nature of nucleation and incorporates the influence of local fluid flow and geometry conditions.

In one example embodiment, a degree of influence in nucleation is computed per geometrical characteristic and flow characteristic in each capillary. These features provide the technical benefit of nucleation models that compute the probability of becoming a nucleation site, incorporating the effect of flow in confined spaces as in porous media, where the local geometry severely affects the fluid flow characteristics and consequent chemical reactions along the fluid paths; and nucleation models for predicting the impact of precipitation on the rock pore geometry that considers the probabilistic nature of nucleation and incorporates the influence of local fluid flow and geometry conditions.

In one example embodiment, an influence weight is computed, per capillary in the rock capillary network 232, due to a combined effect of pore geometric change and flow characteristics, the probability of nucleation based on nucleation models. These features provide the technical benefit of nucleation models that compute the probability of becoming a nucleation site, incorporating the effect of flow in confined spaces as in porous media, where the local geometry severely affects the fluid flow characteristics and consequent chemical reactions along the fluid paths; and nucleation models for predicting the impact of precipitation on the rock pore geometry that considers the probabilistic nature of nucleation and incorporates the influence of local fluid flow and geometry conditions.

In one example embodiment, the identifying the one or more hotspots of nucleation further comprises determining a set of hotspots of nucleation associated with the rock capillary network 232 based on the probability of nucleation being above a given threshold. These features provide the technical benefit of using the probability of nucleation and a threshold to identify the hotspots of nucleation.

In one example embodiment, each instance of the geometry evolution simulation computes the mineral precipitation on a randomly selected subset of capillaries, in the rock capillary network 232, corresponding to the one or more identified hotspots of nucleation. These features provide the technical benefit of measuring the expected rock geometry alterations and associated alterations of the rock properties from an aggregate of results (for example, simulating an ensemble of systems with different (random) initializations and taking the ensemble average as the significant result where some of the rock properties include porosity, permeability, precipitation volume, amount of stored carbon dioxide and the like).

In one example embodiment, an influence of a set of flow characteristics and geometrical characteristics in a mineral precipitation rate is computed per capillary in the rock capillary network 232. These features provide the technical benefit of measuring the expected rock geometry alterations and associated alterations of the rock properties from an aggregate of results (for example, simulating an ensemble of systems with different (random) initializations and taking the ensemble average as the significant result where some of the rock properties include porosity, permeability, precipitation volume, amount of stored carbon dioxide and the like).

In one example embodiment, the performing of each instance of the geometry evolution simulation further comprises determining the initial and boundary conditions, and pore-scale parameters based on materials chemical reactions and physical properties. These features provide the technical benefit of supporting a variety of materials in the simulation.

In one example embodiment, the performance of each instance of the geometry evolution simulation further comprises determining an iteration time interval 276 from a reaction time period necessary to produce a change in a pore geometry equal to or greater than one digital rock discretization size. It is noted that, as a representation of the pore space of the digital rock sample, discretized with a resolution limited by the digital volume element (voxel) size, the CNM can only capture spatial domain changes as small as one voxel size, with smaller increments not producing any alteration to the capillary diameter in the CNM. Setting a time interval in which the material accumulation induces a change in diameter smaller than one voxel will not influence the CNM, as multiple temporal iterations will be required to produce a change. Thus, these features provide the benefit of efficiently running the simulations for a period of time that produces a relevant change in the pore geometry.

In one example embodiment, operations of each instance of the geometry evolution simulation are repeated until a specified criteria is reached, where the specified criteria comprises one or more of a value of porosity, a value of permeability, an amount of accumulated mineral precipitation volume, and an amount of simulation time. These features provide the technical benefit of ceasing a simulation once a specified goal of the simulation has been attained.

Techniques as disclosed herein can thus provide substantial beneficial technical effects. Some embodiments may not have these potential advantages and these potential advantages are not necessarily required of all embodiments. By way of example only and without limitation, one or more embodiments may provide one or more of:
techniques for computing chemical reactions and physical property changes on rock capillaries, by a fluid flow;
a computational workflow that enables the prediction of spatiotemporal geometry evolution of the porous structure, within a capillary network representation of a rock sample;
a system for estimating the volume of carbon dioxide mineralized and stored at a large scale, such as on the scale of a reservoir, based on fluid flow interactions on rock pore-scale;
techniques that address the interactions between changes in the local flow conditions of a porous structure due to geometry evolution and their influence on the physical properties and chemical reactions;
techniques that consider the influence of the local flow conditions at the nucleation onset and the evolution of each simulated process (for example, a rock capillary flow rate can influence the CO₂ nucleation onset and rock capillary diameter rate of erosion);
techniques that address the progression of simultaneous physical and chemical processes within the rock capillary network and the effect of simulating vastly different time scales;
techniques for optimizing the simulation parameters for maximum computational efficiency while retaining accuracy;
techniques for adopting the physical and chemical reaction results to the capillary network representation enabling larger rock sample volumes in simulation with same computing resources;
improving a variety of practical applications, such as carbon dioxide geological sequestration, oil recovery, reservoir engineering, and the like;
supporting the generation of new additive candidates through simulation to accelerate CO₂ mineralization under geological conditions;
nucleation models suitable for application to the entire rock sample (not just flat surfaces, grains, or small portions of porous media), enabling a stochastic study that considers all possible outcomes, which would be impractical when applying existing models on large or complex geometries;
nucleation models that compute the probability of becoming a nucleation site, incorporating the effect of flow in confined spaces as in porous media, where the local geometry severely affects the fluid flow characteristics and consequent chemical reactions along the fluid paths;
nucleation models for predicting the impact of precipitation on the rock pore geometry that considers the probabilistic nature of nucleation and incorporates the influence of local fluid flow and geometry conditions (e.g., locations within the rock pore under backflow, reverse pressure gradients or where geometry shows throats, heels, elbows, and ends of capillaries that will present a higher probability of becoming a hotspot of nucleation);
a process for the computation of the porous geometry evolution that models the probabilistic nature of the nucleation onset to identify a set of most likely mineralization spots, where mineralization spots (referred to as hotspots of nucleation or simply hotspots herein) refers to those rock capillary locations where mineral precipitation will happen;
a system that repeats simulations with different randomly selected sub-sets from a set of the most likely hotspots of nucleation, per rock capillary, and calculates the expected geometry alterations of the rock capillaries from aggregate results. For example, simulating an ensemble of processes with different (random) initializations and taking the ensemble average as the significant result; and
a system readily adapted to parallel Workflow Orchestration where the multiple simulation processes are seamlessly launched to run in parallel on a cloud-computing cluster and the results are aggregated and stored for user analysis through a Representational State Transfer application programming interface (REST API) container.

Generally, techniques are provided for computationally predicting the impact of mineralization within a capillary network representation of a rock porous media. Exemplary embodiments extend conventional methods based on deterministic models. For example, the probabilistic nature of the nucleation onset through the stochastic simulation of mineral precipitation and accumulation under the influence of the fluid flow on the rock capillaries geometry.

In one example embodiment, a stochastic simulation of mineral precipitation and accumulation considers the probabilistic nature of the nucleation onset under the influence of the fluid flow on a capillary geometry. Example embodiments support the materials discovery to calibrate models that will help screen materials for carbon dioxide (CO₂) geological sequestration.

### Stochastic Simulation Method of Mineral Precipitation

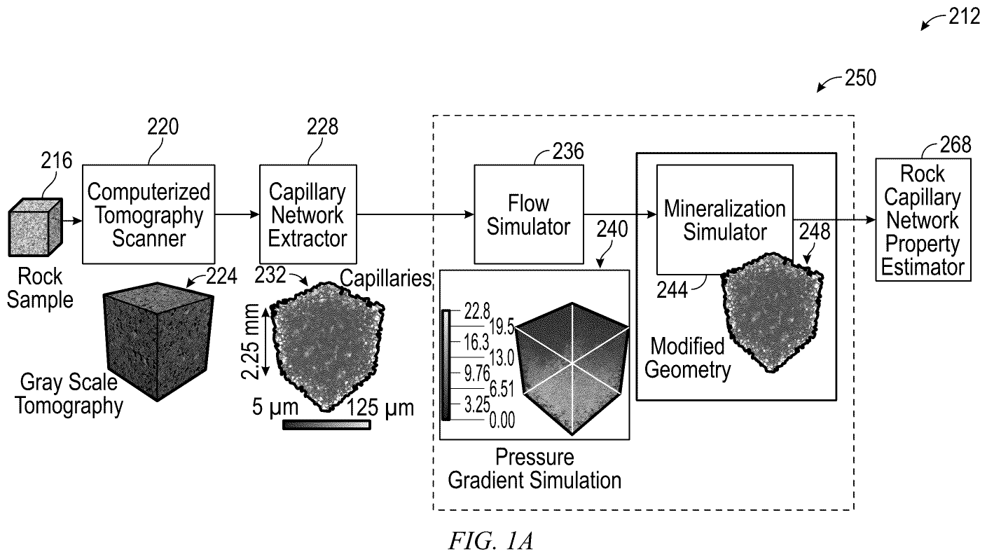

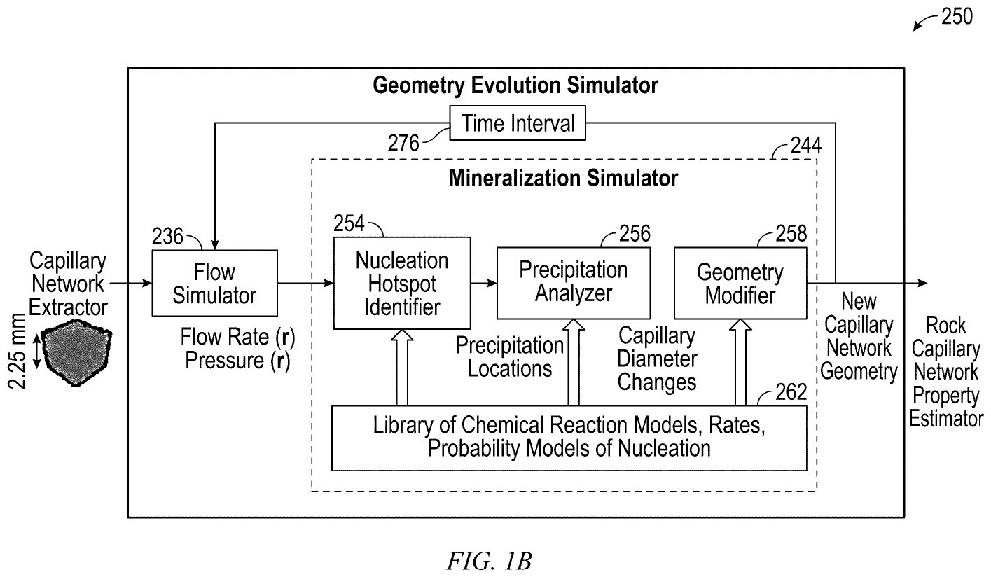

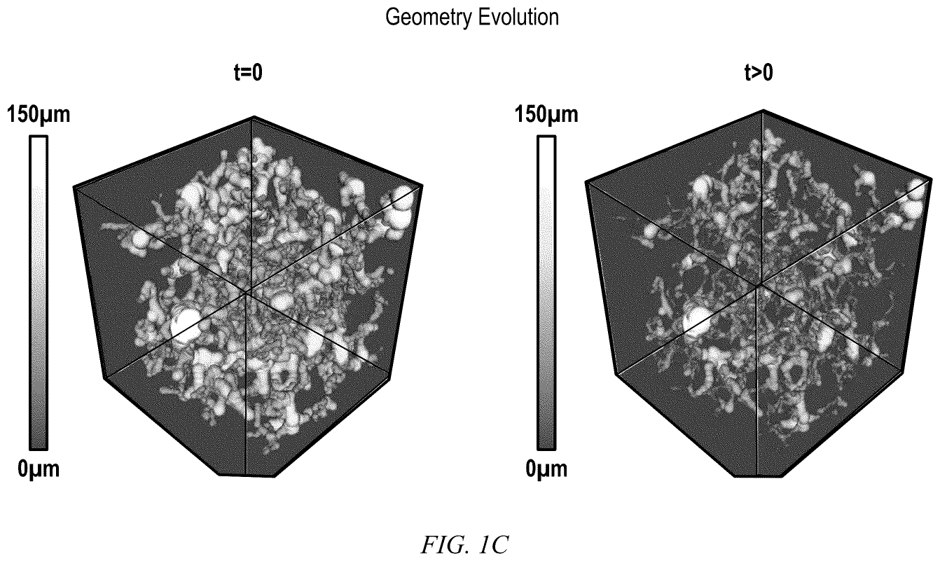

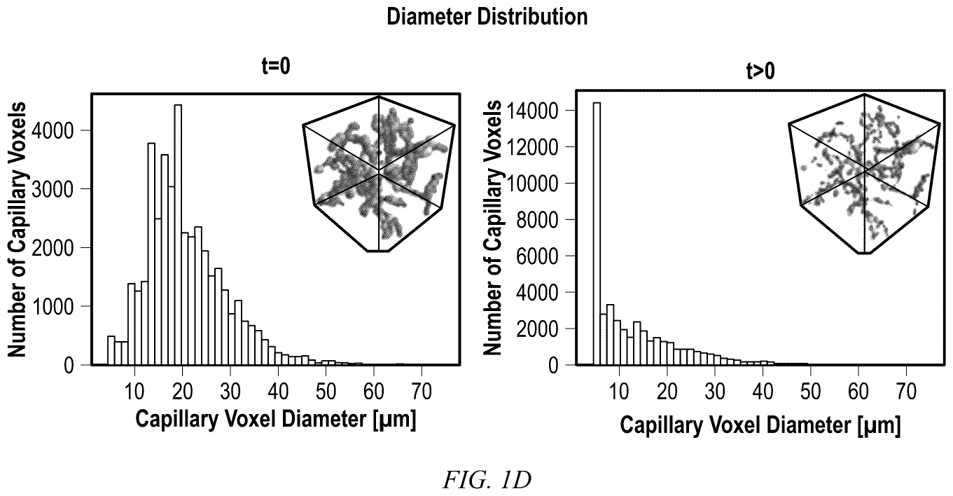

FIG. 1A is a high-level block diagram 212 of an example system for simulation of mineralization in rock porous media by statistical sampling of nucleation sites, in accordance with an example embodiment. In one example embodiment, a computerized tomography (CT) scanner 220 scans a rock sample 216 to generate a grey-scale tomography 224. Rock tomography data may include, for example, one million to one billion volume elements or voxels. A capillary network extractor 228 produces a capillary network model (CNM) geometry 232 of the capillaries of the rock sample 216 based on the grey-scale tomography 224. For example, an image processing method (which may be integrated into the capillary network extractor 228) can be used to convert the grey-scale of the tomography to binary values, to sharpen the captured image, and the like. In one example embodiment, a pore-scale flow simulator 236 performs a pore-scale fluid flow simulation on a porous medium, such as the capillaries of the rock sample 216, where flow is driven by a pressure gradient applied across the capillaries of the rock sample 216 in one of its axes to generate pressure gradient field, flow rate field and the like based on a corresponding capillary network model (CNM) geometry. (See pressure gradient simulation 240.) To simulate both single and two-phase flow through the capillary network model 232 of the pore geometry, laminar flow is assumed and the equations relating pressure and flow are applied within each capillary, followed by conservation of mass at each network node, to build a large system of coupled equations in sparse matrix form. Solving this matrix yields flow properties, like the pressure distribution or flow rate, at each point in the network, from where to extract bulk flow properties like permeability or saturation by one fluid. A mineralization simulator 244 runs a simulation based on the pressure gradient results of the flow simulation to generate a modified capillary geometry 248 that represents the capillary network model (CNM) geometry 232 at a future point in time, such as after a period of precipitation, erosion, and the like. In one example embodiment, the pore-scale flow simulator 236 and the mineralization simulator 244 form a geometry evolution simulator module 250 (also referred to as branches and simulation instances herein), passing the results to the rock capillary network property estimator 268.

FIG. 1B is a high-level block diagram of an example geometry evolution simulator sub-system 250 for computing the impact of fluid-rock processes on the pore scale geometry, in accordance with an example embodiment. The geometry evolution simulator sub-system 250 includes the pore-scale flow simulator 236 and the mineralization simulator 244 (of FIG. 1A). In one example embodiment, the mineralization simulator 244 includes a nucleation hotspot identifier 254, a precipitation analyzer 256, a library of chemical reaction models, rates, probability models of nucleation 262, and a geometry modifier module 258. The nucleation hotspot identifier 254 is introduced in the computation of the porous geometry evolution to model the probabilistic nature of the onset of nucleation where a set of most likely precipitation capillaries (referred to as nucleation hotspots herein) is identified, and a smaller set is randomly selected to compute the impact of crystal accumulation. The precipitation analyzer 256 adjusts the precipitation estimate to the local flow and geometry conditions on each selected nucleation hotspot.

The geometry modifier module 258 of the mineralization simulator 244 produces, using the results of the precipitation analyzer 256 (where the results indicate changes to the capillary diameters due to precipitation, as estimated using a library 262 of chemical reaction models, rates, probability models of nucleation and the like), a revised capillary network model (CNM) geometry based on the produced metrics. FIG. 1C is an example of a porous rock capillary network representation at two instants in a simulation, in accordance with an example embodiment. One representation (left-side of FIG. 1C) corresponds to the beginning of the simulations (at time t=0), and one representation (right-side of FIC. IC) corresponds to the results obtained after a few iterations of the simulation have been executed and some amount of time has elapsed (at time t>0). FIG. 1D is a representation of an example distribution of pore diameters within a rock capillary network representation at two instants in the simulation, in accordance with an example embodiment. One representation (left-side of FIG. 1D) corresponds to the beginning of the simulations (at time t=0), and one representation (right-side of FIC. 1D) corresponds to the results obtained after a few iterations of the simulation have been executed and some amount of time has elapsed (at time t>0).

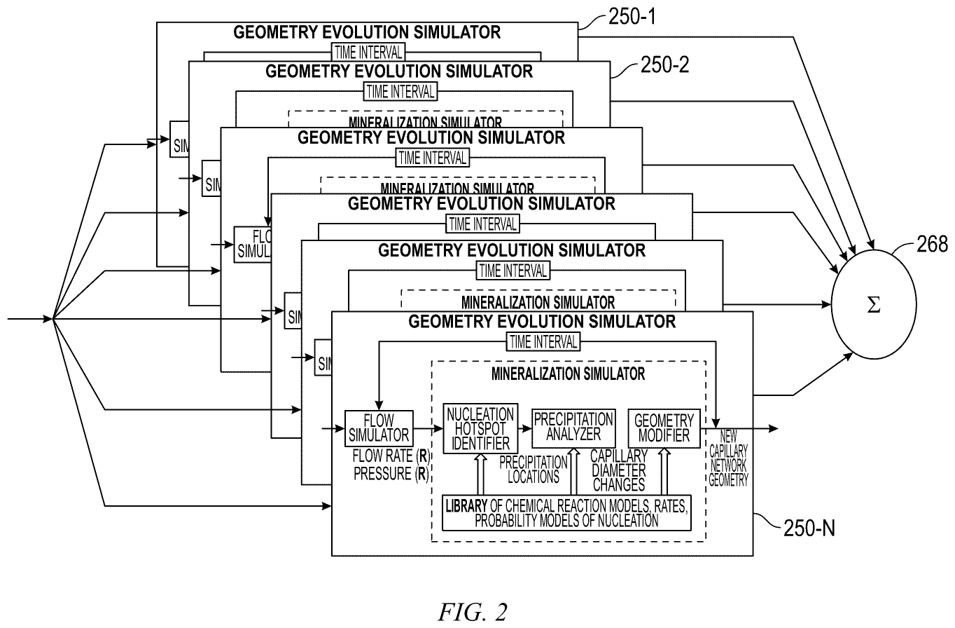

In the workflow of mineralization simulator 244, changes on the network geometry are expected to arise from mineral accumulation on a set of the most likely precipitation spots or capillaries computed on the initial geometry, and the network geometry is updated only on those capillaries by the geometry modifier module 258 during each iteration of the geometry evolution simulator sub-system 250. This approach is repeated in a parallel or serial manner per branch (geometry evolution simulator sub-system) 250-1, 250-2, . . . , 250-N of FIG. 2, each with a randomly selected sub-set from the same global set of nucleation hotspots in the CNM as identified by the nucleation hotspot identifier 254 and their changes in diameter computed by the precipitation analyzer 256 on the initial geometry. In one example embodiment, the subset of nucleation hotspots initially selected for each branch or instance remains constant with each iteration of a corresponding branch (geometry evolution simulator sub-system) 250-1, 250-2, . . . , 250-N, with the geometry modifier module 258 only updating the geometry at the capillaries in that subset. As the simulation progresses, the geometry evolution of each parallel simulation branch 250-1, 250-2, . . . , 250-N diverges from the other branches 250-1, 250-2, . . . , 250-N such that, at time t>0, each parallel realization may have evolved into a completely different geometry. In another example embodiment, the list of nucleation hotspots for the modified geometry is updated with each simulation iteration 250-1, 250-2, . . . , 250-N and a few new nucleation hotspots may be added in each new iteration such that, at time t>0, each branch 250-1, 250-2, . . . , 250-N may have a different set of nucleation hotspots from which to randomly sample and continue to the next iteration. At the end of the simulation, a certain rock property, such as porosity, permeability, accumulated precipitation and the like, is measured in the resulting CNM geometry of each branch 250-1, 250-2, . . . , 250-N. The expected rock geometry alterations and associated alterations of the rock properties as a consequence of the stochastic pore scale reactions is extracted from an aggregate by the rock capillary network property estimator 268 of a plurality of simulation results as the average or weighted average of the final rock property value from all branches 250-1, 250-2, . . . , 250-N.

### Conventional Scenario

Nucleation sites represent locations within the spatial domain where the onset of crystal formation and growth occur (at specific precipitation rates). Nucleation patterns have limited accuracy when predicted using only a deterministic approach (i.e., applying reaction rates equally to all locations in the simulation domain). Conventional solutions propose nucleation models that account for probabilistic effects by, for example, imposing a Gauss-Laplace—normal—probability density function and incorporating them into pore-scale reactive transport solvers. At each position in the domain, nucleation models compute the probability of the location becoming a nucleation site. (A nucleation hotspot is a location within the spatial domain (e.g., one or more capillaries within the capillary network representation of the pore space) with the highest probability of becoming a nucleation site.) However, such conventional models are limited to simple spatial domains like flat surfaces and round grains. They do not incorporate the effect of flow in confined spaces as in porous media, where the local geometry severely affects the fluid flow characteristics and consequent chemical reactions along the fluid paths.

In one example embodiment, the prediction of the impact of precipitation on the rock pore geometry considers the probabilistic nature of nucleation and incorporates the influence of local fluid flow and geometry conditions (e.g., locations within the pore network under backflow, locations within the pore network under reverse pressure gradients, or where the geometry shows throats, heels, elbows, and end of capillaries that will present conditions leading to a higher probability of becoming a nucleation hotspot).

In one example embodiment, the nucleation hotspot identifier 254 and precipitation analyzer 256, described more fully below, identifies precipitation using a library of chemical reaction models, rates, and probability models of nucleation 262 (referred to as library 262 herein).

The geometry modifier module 258 applies the geometry modifications to the CNM due to precipitation. In another example embodiment, the geometry modifier module 258 incorporates the effect of coupled pore-scale phenomena, such as erosion and the like. For example, with information about the time intervals 276 for performing a simulation for each effect and the reaction rates, the change in diameter of each capillary is computed.

FIG. 2 is a high-level block diagram of a plurality of geometry evolution simulator sub-systems 250-1, 250-2, . . . , 250-N and a rock capillary network property estimator 268, in accordance with an example embodiment. The rock capillary network property estimator 268 serves to aggregate the results generated by the plurality of geometry evolution simulator sub-systems 250-1, 250-2, . . . , 250-N. The plurality of geometry evolution simulator sub-systems 250-1, 250-2, . . . , 250-N can be implemented using a plurality of worker nodes that perform the flow simulations and mineralization simulations in a parallel manner, as described more fully below, or by one or more worker nodes that perform at least some of the simulations in a serial manner.

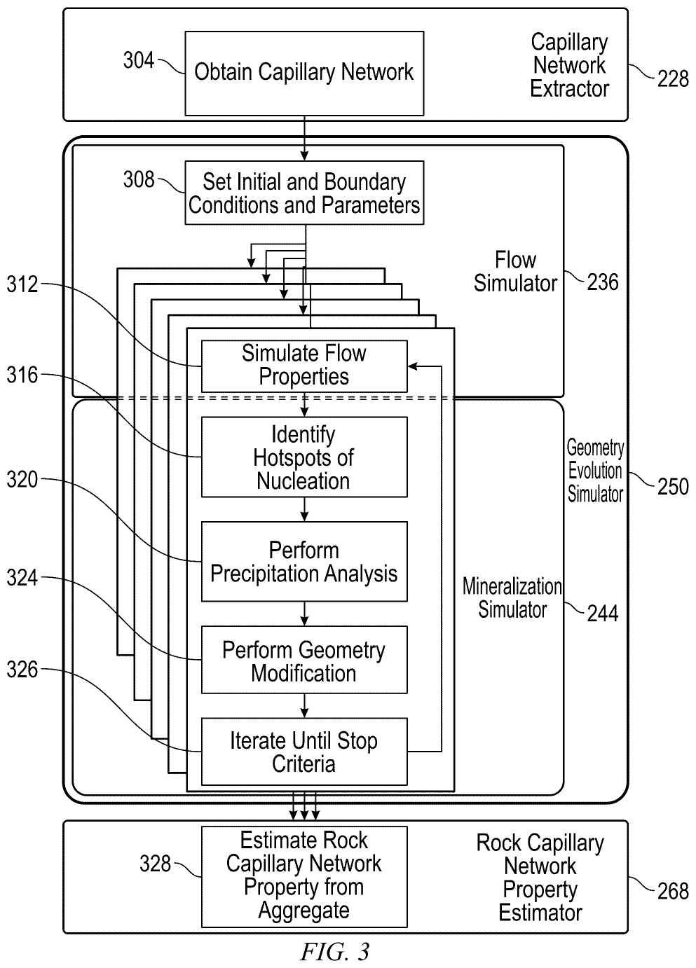

FIG. 3 is a flowchart of an example method for simulation of mineralization in rock porous media by statistical sampling of nucleation sites, in accordance with an example embodiment. In one example embodiment, the method of FIG. 3 considers the influence of local flow and geometry on the onset of nucleation sites. In one example embodiment, a rock capillary network model (CNM) geometry 232 is obtained (operation 304). Initial and boundary conditions of the flow simulation are set and N simulation branches are instantiated (operation 308) by flow simulator 236. Simulation branches may be instantiated serially, in parallel or in a combination of serial and parallel.

The flow simulator 236 performs an iteration of a flow simulation to obtain, for example, fluid flow vectors (operation 312). The nucleation hotspot identifier 254 identifies ‘hotspots’ of nucleation (operation 316) and the precipitation analyzer 256 starts a precipitation analysis and estimates accumulation over a given time interval 276 (operation 320). The geometry modifier module 258 adjusts the capillary network geometry to the effect of precipitation (operation 324). Operations 312-324 are repeated for each instantiation (in either a serial and/or parallel manner) until stop criteria (such as a given number of iterations, a pre-determined value of a rock property or a pre-determined property value change from one iteration to the next) is met (operation 326) and the resulting rock property (such as precipitation volume, porosity, permeability and the like) is output by the corresponding instantiation. In one example embodiment, operations 316-326 are executed by the mineralization simulator 244. The resulting rock sample properties generated by the N instantiations are aggregated together as, for example, the arithmetic average or weighted average of the final property value over the N instances (operation 328) by the rock capillary network property estimator 268. In a non-limiting example, the stop criterion is a change of less than 0.1% (or other predetermined value) of the porosity of the network from one iteration to the next.

### Initial and Boundary Conditions

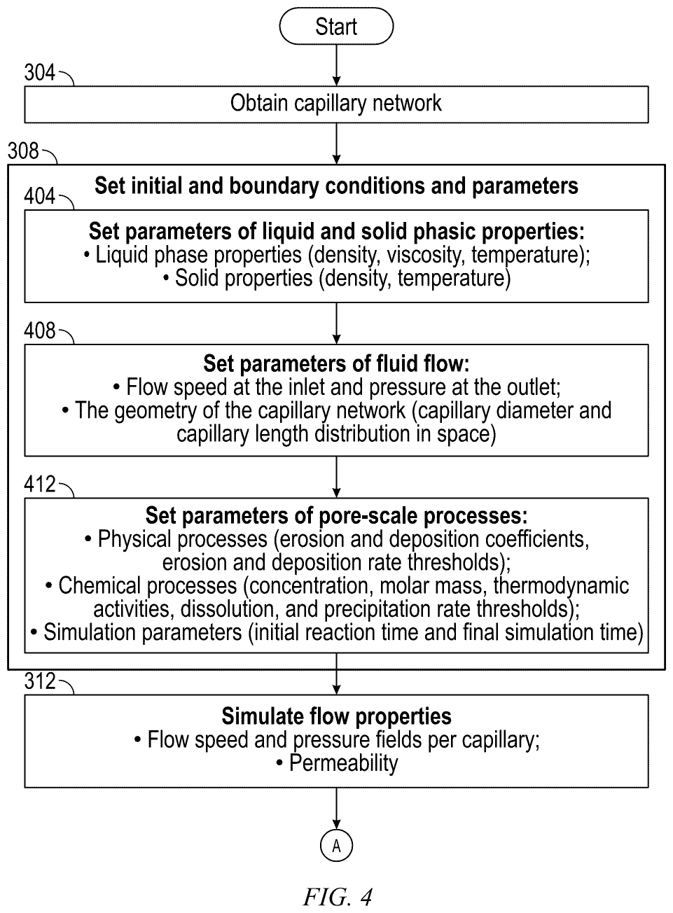

FIG. 4 is a flowchart showing details of operations 304 to 312 of the method of FIG. 3, in accordance with an example embodiment. Given a capillary network model (CNM) geometry 232, the file that contains information on the geometry of each capillary (such as a description of the nodes and the links of the capillary network) is accessed (operation 304). During the establishment of the initial and boundary conditions (operation 308), parameters related to phasic properties of the fluid and solid, the pore-scale processes, and initial and boundary conditions on the fluid flow are imposed prior to running the flow simulation (operation 312) of the flow simulator 236 to obtain the velocity and pressure fields at the steady state. In particular, parameters of liquid and solid phasic properties are set (operation 404), including liquid phase properties (such as density, viscosity, temperature, and the like); and solid matrix properties (such as density, temperature and the like). Initial and boundary conditions of fluid flow are set (operations 408), including flow speed at the inlet and pressure at the outlet; and the geometry of the capillary network model (CNM) geometry 232 (capillary diameter and capillary length distribution in space). Parameters of pore-scale processes are set (operation 412), including physical processes (erosion and deposition coefficients, erosion and deposition rate thresholds and the like); chemical processes (concentration, molar mass, thermodynamic activities, dissolution, precipitation rate thresholds and the like); and simulation parameters (initial reaction time, final simulation time and the like). The flow simulator 236 is run to compute the steady state at each capillary, including pressure gradient, flow speed, permeability, and the like (operation 312). Operation 316 is discussed below.

### Identification of Hotspots of Nucleation

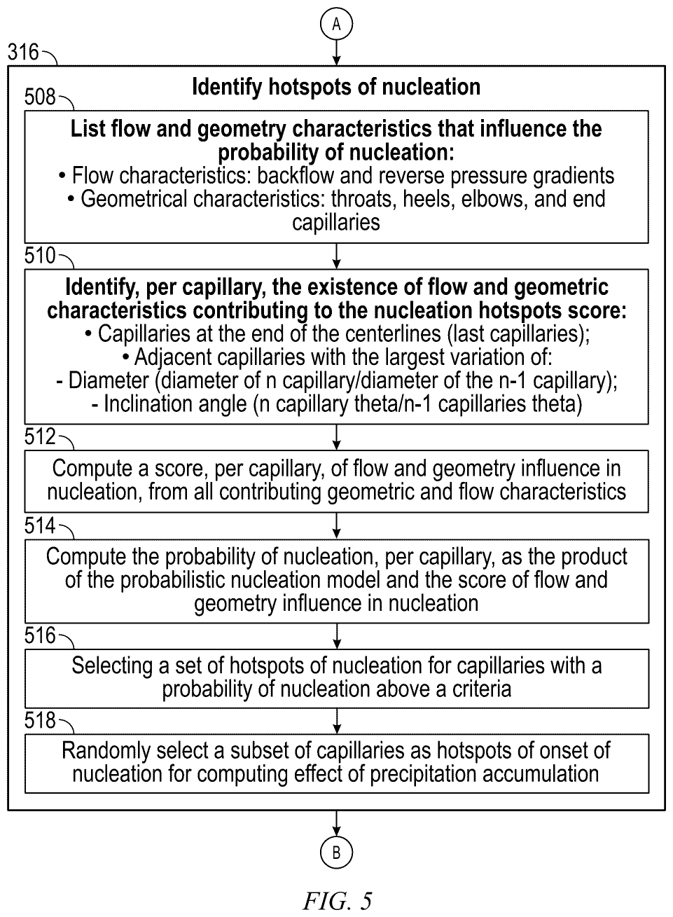

FIG. 5 is a flowchart showing details of operation 316 of the method of FIG. 3, in accordance with an example embodiment. Following the running of the flow simulator 236 to compute the flow properties in the capillary network model (CNM) geometry 232 (operation 312), the hotspots of nucleation are identified (operation 316) by the nucleation hotspot identifier 254. In one example embodiment, to identify the hotspots of nucleation, the following operations are performed:
create a list of flow properties (e.g., backflow and reverse pressure gradients) and geometric elements (e.g., throats, heels, elbows, and end capillaries) that influence the chance of becoming nucleation hotspots within the capillary network (operation 508);
determine, per capillary in the capillary network model (CNM) geometry 232, the presence or absence of each of the flow properties and geometric elements identified as contributing to the probability of the capillary becoming a nucleation hotspot within the capillary network (operation 510), including capillaries at the end of the network (last capillaries) and adjacent capillaries with the largest variation of diameter (throats—diameter of n capillary/diameter of the n−1 capillary) and inclination angle (elbows—n capillary theta/n−1 capillary theta);
assigning, per characteristic, a degree of influence in nucleation (operation 510). For example, if backflow is present, if a substantial diameter change is present, or if there is a significant change in the direction of capillary orientation vs. the previous capillary, the weight assignment of geometrical and flow influence per capillary in the network is computed; and an influencing weight. Given the teachings herein, the skilled artisan can determine a suitable influencing weight for a desired application from heuristics, experimental evidence, simulation and/or literature. The weight assignment may be, for example, a qualitative assignment (1=backflow present and 0=backflow not present); or a quantitative weight assignment based on a probabilistic function, Prob=f(diameter gradient);
compute, per capillary in the network, a score of the degree of influence in nucleation (operation 512) from all contributing geometric and flow characteristics, to be applied as an adequate (flow and geometry) influence weight for the probability of nucleation in each capillary of the network. This score can, for example, be computed as the aggregate of all contributing features present (i.e. those with presence in capillary=1);
compute, per capillary, the probability of nucleation (operation 514) as the product of the probabilistic nucleation model and the score of flow and geometry influence in nucleation from step 512. Given the teachings herein, the skilled artisan can extract a suitable probabilistic nucleation model from the literature, heuristics, or from experimental data of the pore-scale process; this model incorporates the effect of physical variables, such as saturation ratio, nucleation rates or temperature;
selecting a set of hotspots of nucleation, including selecting those capillaries with a probability of nucleation above a specified criteria (operation 516) followed by the random selection, from the capillaries in the selected list, of a subset of capillaries as hotspots of onset of nucleation (operation 518) for computing an effect of precipitation accumulation;
Following the identification of hotspots of nucleation in each parallel simulation, the precipitation analysis (operation 320) is performed by the precipitation analyzer 256.

### Precipitation Analysis

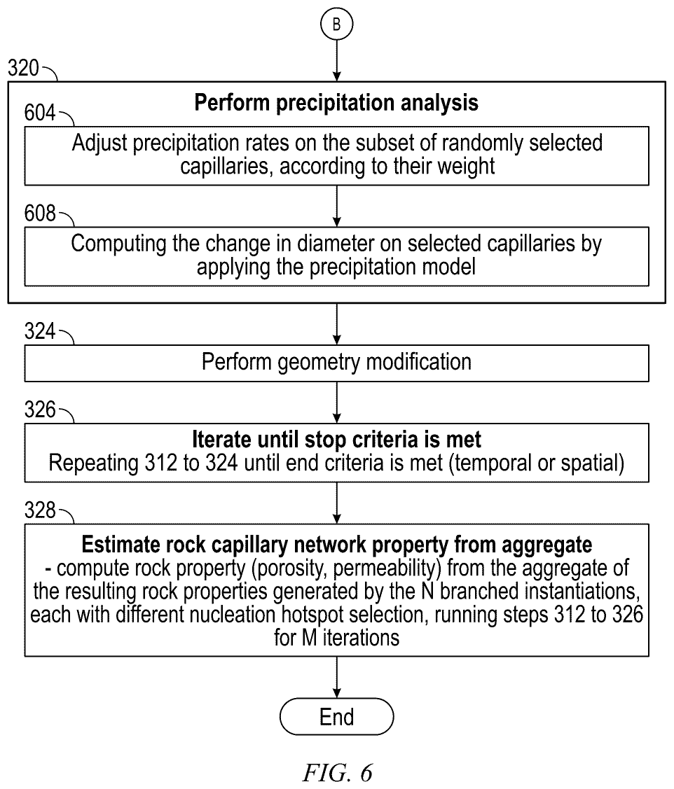

FIG. 6 is a flowchart showing details of operations 320, 324, 326 and 328 of the method of FIG. 3, in accordance with an example embodiment. As noted above, following the identification of hotspots of nucleation, the precipitation analysis (operation 320) is performed, including adjusting precipitation rates per capillary according to their weight (operation 604) which may be determined by the skilled artisan, given the teachings herein, from experimental evidence, heuristics, simulation or literature when available (for instance, the precipitation rate may be dependent on the local flow rate or on abrupt changes in capillary diameter, so the rate of precipitation is adjusted per capillary in each iteration in the form of a variable weighting factor); and computing the change in diameter on those selected hotspots of nucleation by applying the precipitation model and adjusted rate (operation 608).

During operation 324 (perform geometry modification by inserting the effect of precipitation on the CNM model), the diameters of the affected capillaries in the geometry of the porous structure are adjusted and a new CNM file is generated for the next iteration. In operation 326, the criteria for stopping the simulations is checked and if not yet met (temporal or spatial), then operations 312 to 324 are repeated.

The resulting rock sample property (porosity, permeability, precipitation volume, stored carbon dioxide (CO₂) and the like) is computed from the aggregate of N results from the N branched simulation instances 250-1, 250-2, . . . , 250-N (operation 328), each with a different nucleation hotspot selection on the same rock capillary network representation 232 of the rock sample 216, each repeating, M times, steps 312 to 324.

### Rock Sample Capillary Network

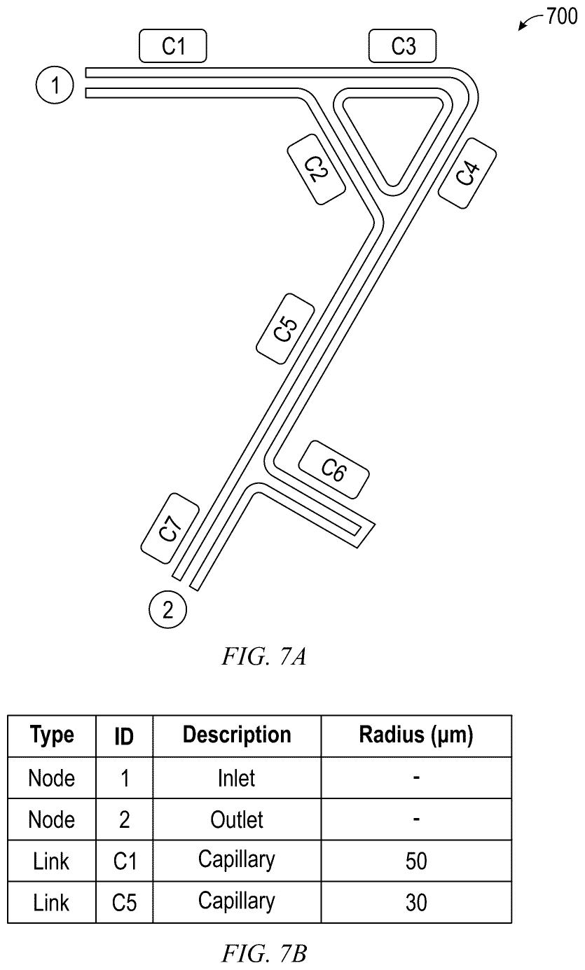

FIG. 7A is a representation of a rock capillary network 700, in accordance with an example embodiment. FIG. 7B is a table providing details regarding the nodes of FIG. 7A and the links (capillaries) between the nodes of FIG. 7A, in accordance with an example embodiment. As illustrated in FIGS. 7A and 7B, node 1 is an inlet node, node 2 is an outlet node and capillaries C1 through C7 have varying diameters, as described in the table of FIG. 7B.

### Performing Flow Simulations to Obtain Fluid Flow Vectors

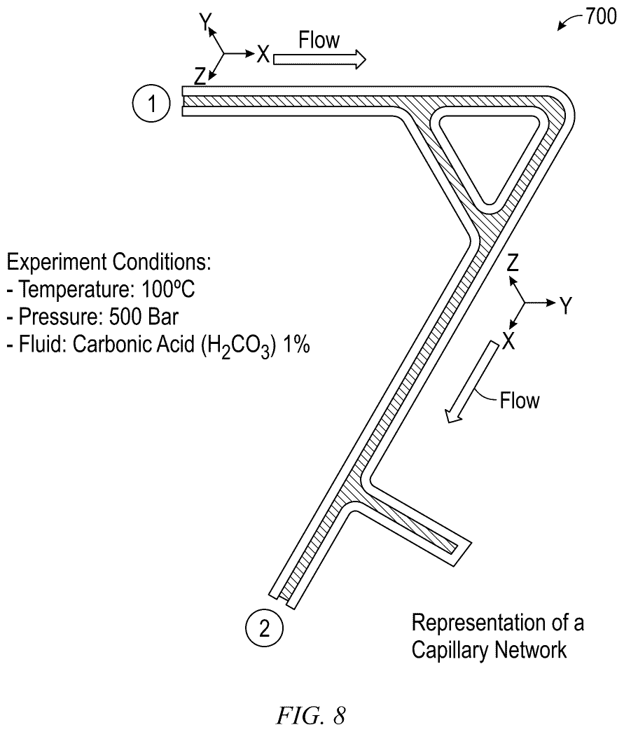

FIG. 8 is a detailed representation of the rock capillary network 700 illustrating the flow from node 1 to node 2, in accordance with an example embodiment. The simulations of FIG. 8 assumed a temperature of 100 degrees Centigrade, a pressure of 500 bars and the fluid was carbonic acid (H₂CO₃) 1%.

Identifying ‘hotspots’ of Nucleation

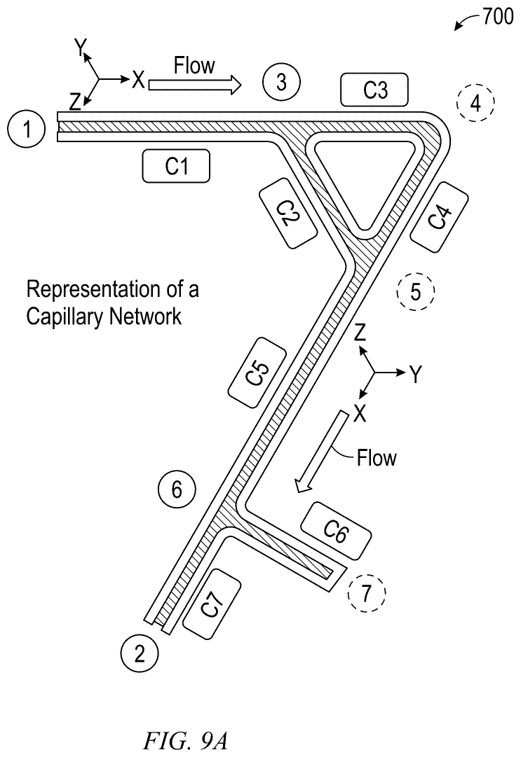

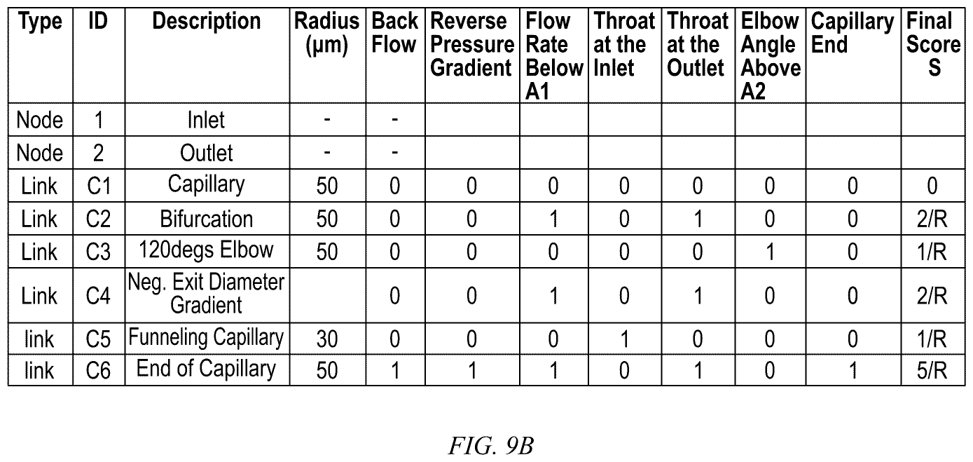

FIG. 9A is a detailed representation of the rock capillary network 700 illustrating the location of nodes 3-7 and the flow from node 1 to node 2, in accordance with an example embodiment. FIG. 9B is a table showing details of the nodes and links (capillaries) within the rock capillary network 700, in accordance with an example embodiment. (Blank entries represent parameters that are not applicable to the corresponding element type, such as a node.) A flow/geometry-based score is combined with probabilistic nucleation models that incorporate the effect of physical variables, such as saturation ratio, nucleation rates or temperature, and the like. The score serves to incorporate the influence of geometry and local flow conditions of the capillary network 700. As illustrated in FIG. 9B, the final score of link C1 is zero, the final score of link C2 is 2/R, the final score of link C3 is 1/R and so on. A set of R flow and geometrical characteristics are evaluated per capillary in the network. These characteristics are evaluated with a binary score indicating its presence (score=1) or absence (score=0). For instance, characteristics, such as whether the capillary is currently sustaining ‘back flow’, ‘reverse pressure gradient’, or includes the presence of ‘throat at the inlet’ or a ‘throat at the outlet’, may be considered. As an example, capillary C5 in the network of FIG. 9A scores 1 for characteristic ‘throat at the inlet’ but 0 for all others in the list. As a result, capillary C5 receives a final score of 1/R. Capillary C6, on the other hand, receives a final score of 5/R due to the presence of ‘back flow’, ‘reverse pressure gradient’, ‘flow rate below A1’, ‘throat at the outlet’ and ‘capillary end.’

### Starting Precipitation and Estimating Accumulation Over a Time Interval

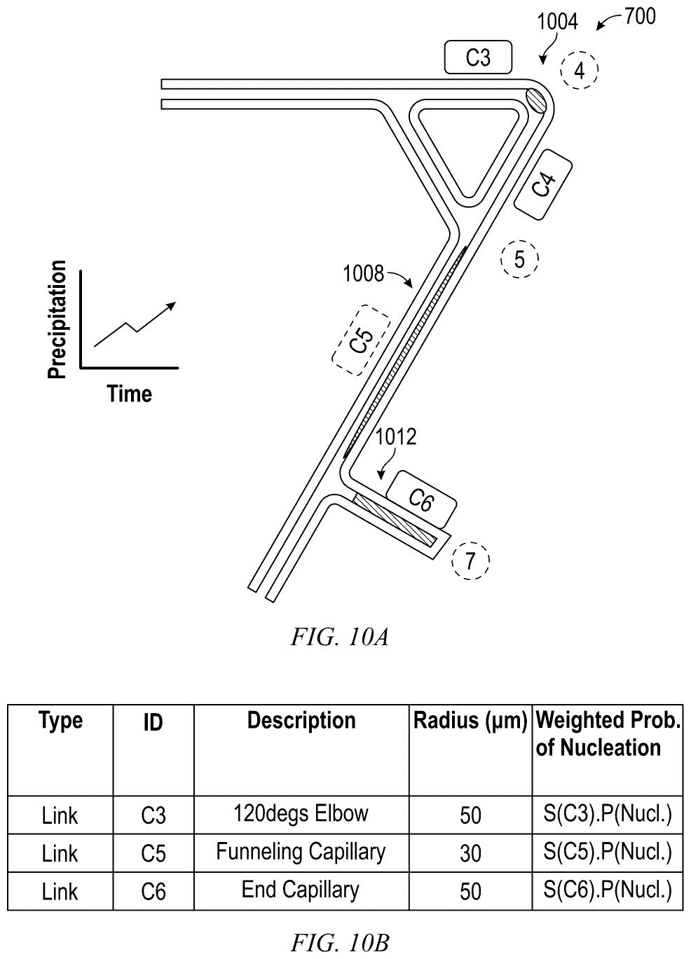

FIG. 10A illustrates example randomly selected nucleation hotspots in the rock capillary network 700, in accordance with an example embodiment. FIG. 10B is a table providing details for the randomly selected nucleation hotspots of FIG. 10A, in accordance with an example embodiment. The example randomly selected hotspots include link 1004 (a 120 degree elbow), link 1008 (a funneling capillary) and link 1012 (an end of capillary).

### Hotspots Identification

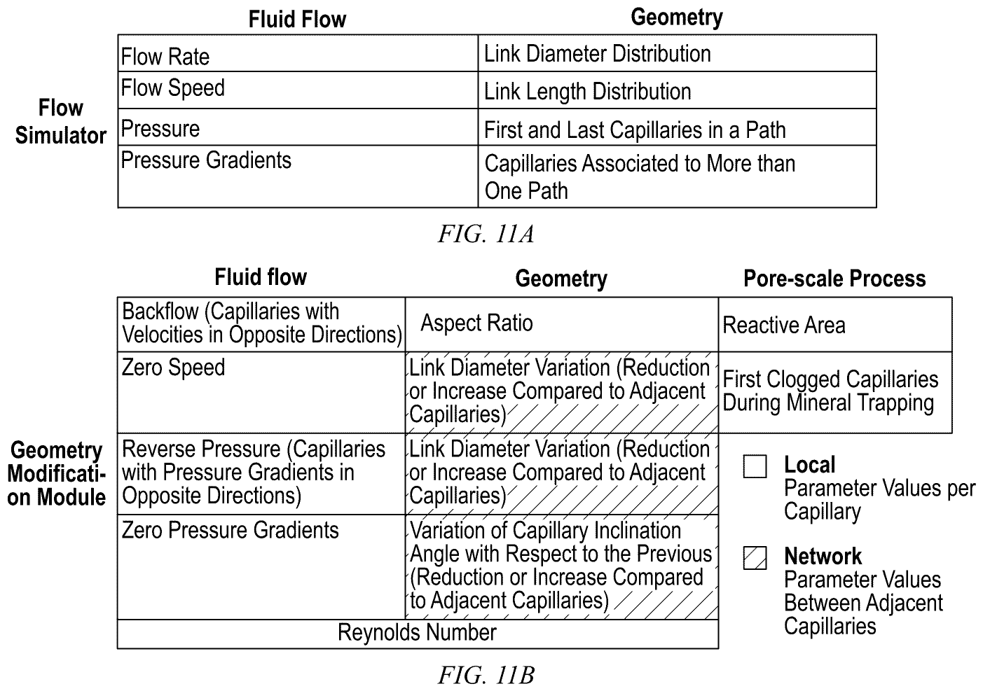

FIGS. 11A and 11B are tables providing details regarding fluid flow, geometry, and pore-scale process parameters that can trigger the onset of nucleation sites within the rock capillary network 700, in accordance with an example embodiment. Flow discovery includes fluid flow, such as flow rate, flow speed, pressure and pressure gradients, and geometry, such as link diameter distribution, link length distribution, first and last capillaries in a path and capillaries associated with more than one path.

### Use Case #1

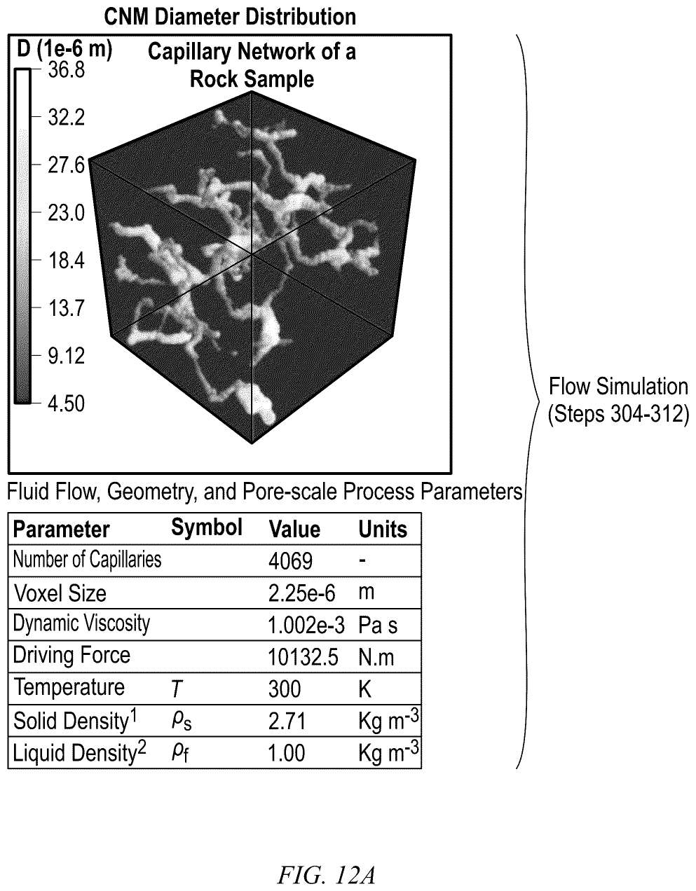

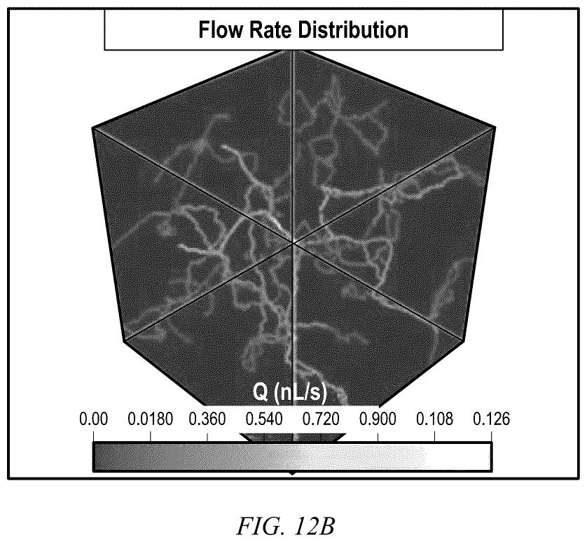

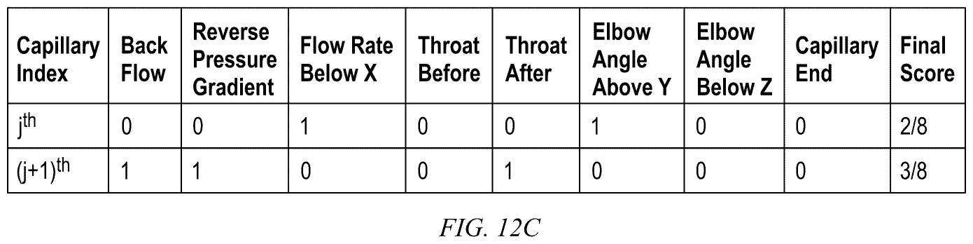

FIGS. 12A-12B illustrate how to estimate the influence of geometrical nucleation hotspots on the flow rate and consequent onset of nucleation and subsequent mineral precipitation, in accordance with an example embodiment. FIG. 12A illustrates a representation of the rock capillary network and parameters used to simulate the physical properties and chemical reactions of fluid flow on capillaries (steps 304 to 312). FIG. 12B displays a resulting flow rate field at every point in the CNM after running the flow simulation.

FIG. 12C is a table illustrating fluid flow and rock capillary geometrical characterization (steps 508-512), in accordance with an example embodiment. Throats and elbows (crosshatched spots) within the capillary network 700 may lead to the onset of nucleation points and accumulation of material during mineral deposition. As illustrated in FIG. 12C, the final score of the j^{th} capillary is 2/8 and the final score of the (j+1)^{th} capillary is 3/8, for a set of 8 geometrical and flow characteristics being monitored.

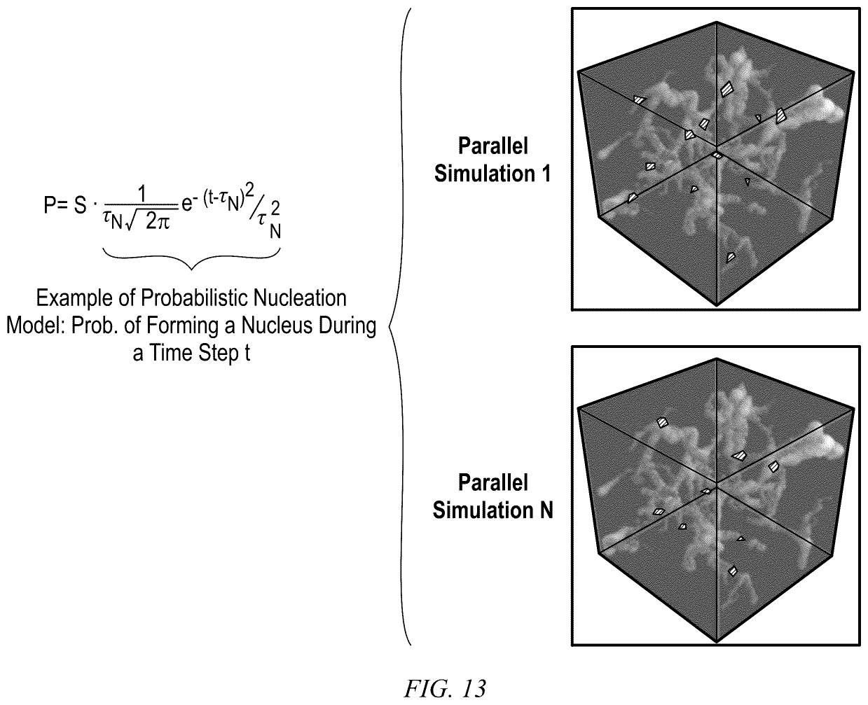

FIG. 13 illustrates two parallel branches of simulation instances, each with a randomly selected sub-set of nucleation hotspots where mineral precipitation is assumed to occur and where the capillary geometry of the rock capillary network is changed, in accordance with an example embodiment. The probability of nucleation per capillary, weighted by the flow and geometrical score S, is calculated (operation 514). A group of hotspots among those of highest probability of nucleation is randomly selected (operations 516 and 518) per each of the parallel threads or branches 250-1, 250-2, . . . , 250-N. The geometrical evolution of each parallel thread is iteratively simulated (operations 312-324) and the hydrodynamic rock property is computed from an aggregate of results (operation 328).

As will be appreciated by the skilled artisan given the teachings herein, an example of probabilistic nucleation model can be extracted from the published literature on probabilistic nucleation as it relates to mineral precipitation and geometry evolution in the porous medium where the probability of forming a nucleus during a time step t is given by the following expression:

$$P ( t ) = S \cdot \frac{1}{{τ}_{N} \sqrt{π}} {e}^{- 8 {( t - {τ}_{N} )}^{2} / {τ}_{N}^{2}}$$

where P is the probability, S is the score of flow and geometry influence in nucleation and τ_{N} is a deterministic mean induction time, deduced through correlation to data obtained experimentally, and that, in an exemplary case, is given as a function of temperature, saturation, interfacial energy, and other parameters.

### Use Case #2

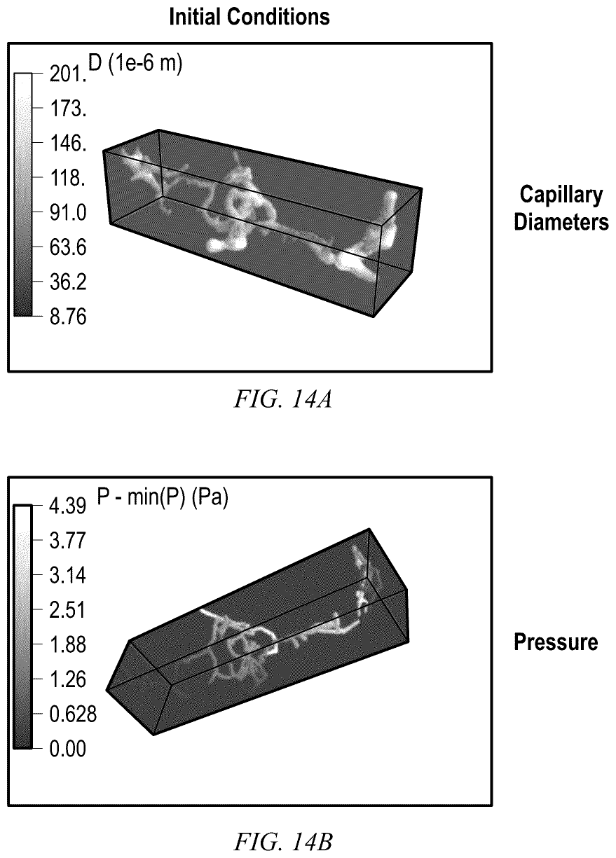

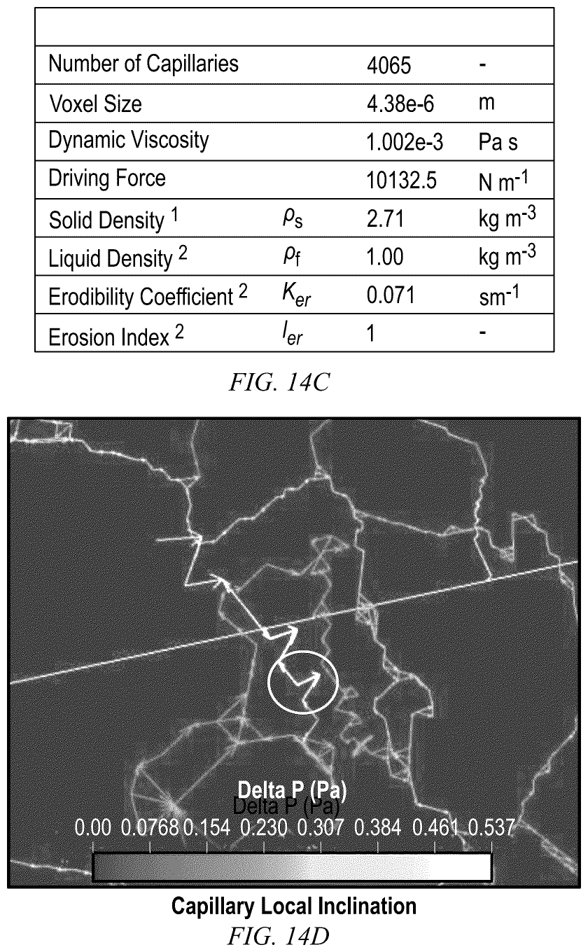

FIG. 14A is a three-dimensional rendering illustrating the typical rock capillary diameter values encountered during the flow simulation, used as an example to show the geometrical characteristics of the capillaries in determining nucleation hotspots leading to consequent onset mineralization, in accordance with an example embodiment.

FIG. 14B is a three-dimensional rendering illustrating the typical pressure values on rock capillaries encountered during the flow simulations, showing the influence of pressure fields on the rock capillaries and in determining hotspots of nucleation leading to the consequent onset of mineralization, in accordance with an example embodiment. According to the flow simulation, 156 capillaries with pressure gradient ΔP≈0 may lead to nucleation sites and the consequent onset of precipitation.

FIG. 14C is a table illustrating an example of flow simulation parameters on the capillary network 700, including fluid flow and capillary geometry, in accordance with an example embodiment.

FIG. 14D is a three-dimensional representation of the capillary local inclination, in accordance with an example embodiment.

### Architecture

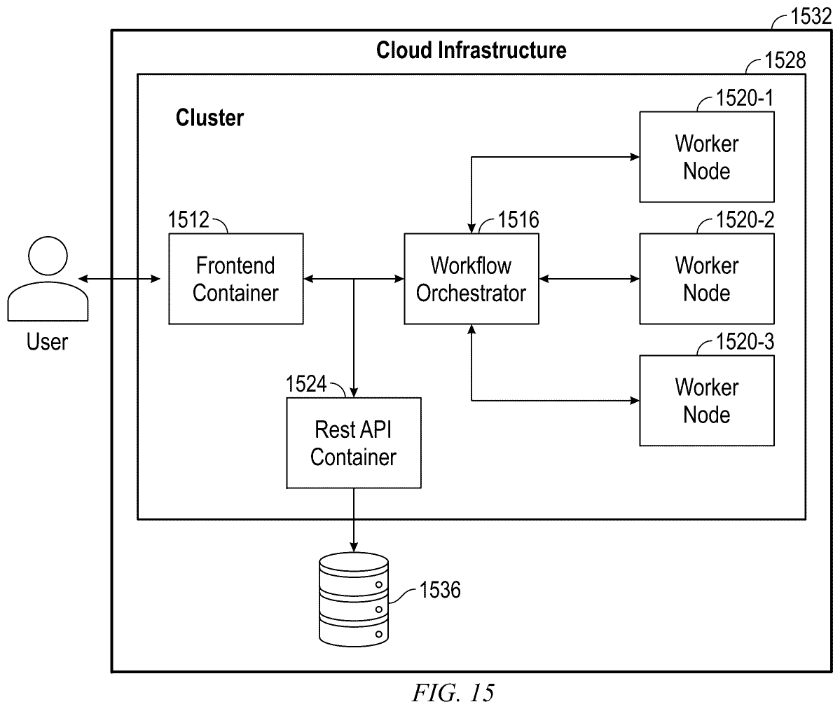

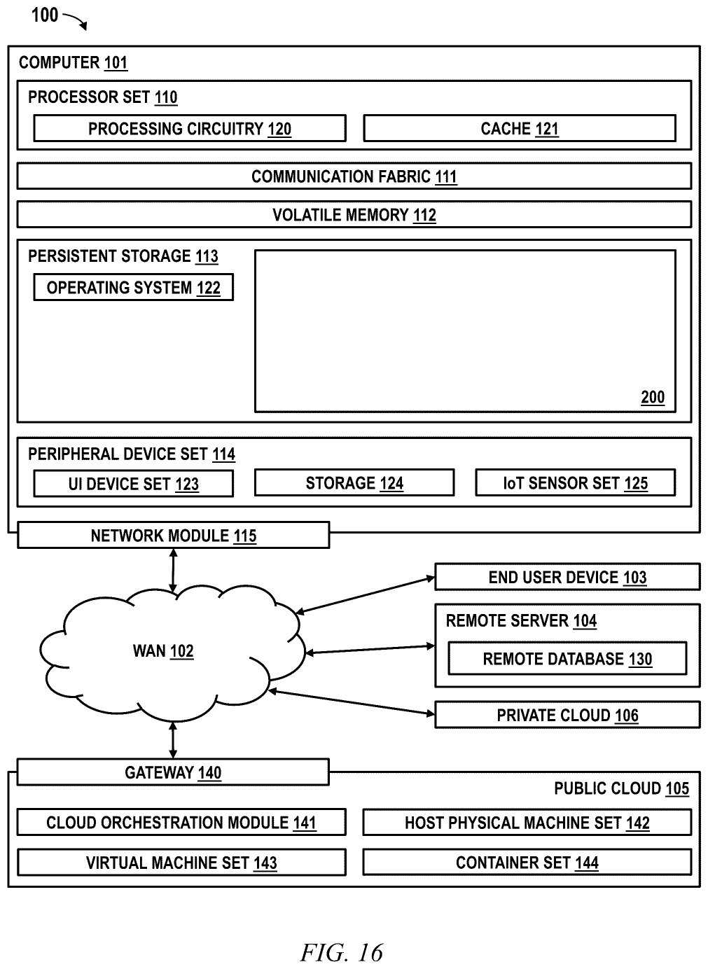

FIG. 15 is an overview of an example system architecture for performing simulation of mineral precipitation in rock porous media, in accordance with an example embodiment. The user interacts with the application through a front-end container 1512 and provides user inputs (such as inputs associated with operations 304 and 308). The front-end container 1512 uses a workflow orchestrator 1516 to launch multiple simulations (operations 312-326) on a cloud-computing cluster 1528 having multiple worker nodes 1520-1, 1520-2, 1520-3. The sample properties (328) resulting from these simulations are stored in a database 1536 hosted on a cloud infrastructure 1532. The Representational State Transfer application programming interface (REST API) container 1524 provides web endpoints for modifying and querying the database from the front-end container 1512 and for retrieving the results (328). An exemplary cloud environment is discussed below with respect to FIG. 16.

### Refer Now to FIG. 16.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as mineralization simulation system 200. Further, one or more embodiments, as noted, can improve a variety of practical applications such as reservoir engineering (a branch of petroleum engineering that applies scientific principles to the fluid flow through a porous medium during the development and production of oil and gas reservoirs so as to obtain a high economic recovery), oil recovery, and/or carbon dioxide geological sequestration. Thus, based on simulations herein, control signals could be sent (e.g., over WAN 102) to operate an oil field (drills, valves, or other oil field equipment, etc.)(e.g., in accordance with reservoir engineering principles), sequester carbon dioxide with carbon dioxide sequestration equipment, or the like. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 16. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for performing a stochastic simulation of mineral precipitation and mineral accumulation on a rock pore-scale comprising:
obtaining a representation of a rock capillary network;
setting initial and boundary conditions of fluid flow and mineral precipitation process simulations for the rock capillary network;
performing one or more instances of a geometry evolution simulation, each geometry evolution simulation comprising:
obtaining fluid flow vectors for the rock capillary network;
identifying one or more hotspots of nucleation in the rock capillary network;
starting a mineral precipitation analysis and estimating the mineral accumulation over a given time interval for at least the identified hotspots of nucleation;
adjusting a pore geometry to an effect of mineral precipitation for the rock capillary network; and
iteratively repeating each of the one or more geometry evolution simulations until corresponding stop criteria are met for each geometry evolution simulation; and
computing a resulting rock under analysis property from an aggregate of results of the one or more geometry evolution simulations.

2. The method of claim 1, further comprising computing an amount of carbon dioxide mineralized and stored in a corresponding rock capillary network representation.

3. The method of claim 1, further comprising computing estimates of converted and stored carbon dioxide in a rock under analysis.

4. The method of claim 1, further comprising computing, per capillary in the rock capillary network, a set of flow characteristics and geometrical characteristics that influence a probability of nucleation.

5. The method of claim 4, further comprising computing, per geometrical characteristic and flow characteristic in each capillary, a degree of influence in nucleation.

6. The method of claim 5, further comprising computing, per capillary in the rock capillary network, an influence weight, due to a combined effect of pore geometric change and flow characteristics, the probability of nucleation based on nucleation models.

7. The method of claim 6, wherein the identifying the one or more hotspots of nucleation further comprises determining a set of hotspots of nucleation associated with the rock capillary network based on the probability of nucleation being above a given threshold.

8. The method of claim 1, wherein each instance of the geometry evolution simulation computes the mineral precipitation on a randomly selected subset of capillaries, in the rock capillary network, corresponding to the one or more identified hotspots of nucleation.

9. The method of claim 1, further comprising computing, per capillary in the rock capillary network, an influence of a set of flow characteristics and geometrical characteristics in a mineral precipitation rate.

10. The method of claim 1, wherein the performing of each instance of the geometry evolution simulation further comprises determining the initial and boundary conditions, and pore-scale parameters based on materials chemical reactions and physical properties.

11. The method of claim 1, wherein the performing of each instance of the geometry evolution simulation further comprises determining an iteration time interval from a reaction time period necessary to produce a change in a pore geometry equal to or greater than one digital rock discretization size.

12. The method of claim 1, further comprising repeating operations of each instance of the geometry evolution simulation until a specified criteria is reached, where the specified criteria comprises one or more of a value of porosity, a value of permeability, an amount of accumulated mineral precipitation volume, and an amount of simulation time.

13. A computer program product, comprising:
one or more tangible computer-readable storage media and program instructions stored on at least one of the one or more tangible computer-readable storage media, the program instructions executable by a processor, the program instructions comprising:
obtaining a representation of a rock capillary network;
setting initial and boundary conditions of fluid flow and mineral precipitation process simulations for the rock capillary network;
performing one or more instances of a geometry evolution simulation, each geometry evolution simulation comprising:
obtaining fluid flow vectors for the rock capillary network;
identifying one or more hotspots of nucleation in the rock capillary network;
starting a mineral precipitation analysis and estimating a mineral accumulation over a given time interval for at least the identified hotspots of nucleation;
adjusting a pore geometry to an effect of mineral precipitation for the rock capillary network; and
iteratively repeating each of the one or more geometry evolution simulations until corresponding stop criteria are met for each geometry evolution simulation; and
computing a resulting rock under analysis property from an aggregate of results of the one or more geometry evolution simulations.

14. A system comprising:
a memory; and
at least one processor, coupled to said memory, and operative to perform operations comprising:
obtaining a representation of a rock capillary network;
setting initial and boundary conditions of fluid flow and mineral precipitation process simulations for the rock capillary network;
performing one or more instances of a geometry evolution simulation, each geometry evolution simulation comprising:
obtaining fluid flow vectors for the rock capillary network;
identifying one or more hotspots of nucleation in the rock capillary network;
starting a mineral precipitation analysis and estimating a mineral accumulation over a given time interval for at least the identified hotspots of nucleation;
adjusting a pore geometry to an effect of mineral precipitation for the rock capillary network; and
iteratively repeating each of the one or more geometry evolution simulations until corresponding stop criteria are met for each geometry evolution simulation; and
computing a resulting rock under analysis property from an aggregate of results of the one or more geometry evolution simulations.

15. The system of claim 14, wherein the at least one processor is further operative to compute an amount of carbon dioxide mineralized and stored in a corresponding rock capillary network representation.

16. The system of claim 14, wherein the at least one processor is further operative to compute estimates of converted and stored carbon dioxide in a rock under analysis.

17. The system of claim 14, wherein the at least one processor is further operative to compute, per capillary in the rock capillary network, a set of flow characteristics and geometrical characteristics that influence a probability of nucleation.

18. The system of claim 17, wherein the at least one processor is further operative to compute, per geometrical characteristic and flow characteristic in each capillary, a degree of influence in nucleation.

19. The system of claim 18, wherein the at least one processor is further operative to compute, per capillary in the rock capillary network, an influence weight, due to a combined effect of pore geometric change and flow characteristics, the probability of nucleation based on nucleation models.

20. The system of claim 19, wherein the identifying of the one or more hotspots of nucleation further comprises determining a set of hotspots of nucleation associated with the rock capillary network based on the probability of nucleation being above a given threshold.
